(19) United States
(12) Patent Application Publication
(10) Pub. No.: US 2020/0281879 A1
(43) Pub. Date: Sep. 10, 2020

(54) STABLE LIQUID COMPOSITION OF KETOPROFEN, SALTS AND ENANTIOMERS THEREOF

(71) Applicant: AZIENDE CHIMICHE RIUNITE ANGELINI FRANCESCO A.C.R.A.F. S.p.A., Roma (IT)

(72) Inventors: Danilo D'AMICO, Cerro la Volturno (IS) (IT); Leonardo MARCHITTO, Porto Recanati (MC) (IT); Lorella RAGNI, Chiaravalle (AN) (IT); Luca DONATI, Porto San Giorgio (AP) (IT); Vincenzo RUSSO, Aprilia (LT) (IT)

(73) Assignee: AZIENDE CHIMICHE RIUNITE ANGELINI FRANCESCO A.C.R.A.F. S.p.A., Roma (IT)

(21) Appl. No.: 16/764,624

(22) PCT Filed: Nov. 28, 2018
(86) PCT No.: PCT/EP2018/082754

§ 371 Date: May 15, 2020

(30) Foreign Application Priority Data
Nov. 30, 2017 (EP) 17204585.8

(51) Int. Cl. A61K 31/192; A61K 47/40; A61K 47/18; A61K 47/42; A61K 47/12; A61K 47/36; A61K 9/08; A61K 9/06

(52) U.S. Cl. CPC A61K 31/192; A61K 47/40; A61K 47/18; A61K 9/06; A61K 47/12; A61K 47/36; A61K 9/08; A61K 47/42

(57) ABSTRACT

A liquid pharmaceutical composition for oral administration comprising a complex of ketoprofen, dexketoprofen or their salts, a β-cyclodextrin and a hydroxyalkylamine, having good palatability and improved chemico-physical and microbiological stability.

### FIELD OF THE INVENTION

The present invention relates to a liquid pharmaceutical composition for oral administration comprising a complex of ketoprofen, a β-cyclodextrin and a hydroxyalkylamine, having good palatability and improved chemico-physical and microbiological stability.

### BACKGROUND OF THE INVENTION

The term Nonsteroidal Anti-Inflammatory Drugs (NSAIDs) is used to indicate a group of molecules able to provide combined analgesic, antipyretic, and anti-inflammatory effects.

Said effects are due to the nonselective inhibition of both cyclooxygenase-1 (COX-1) and cyclooxygenase-2 (COX-2) isoenzymes, which catalyse the formation of prostaglandins and thromboxane from arachidonic acid. Prostaglandins act, among other things, as messenger molecules in the process of inflammation and hyperalgesia, and are triggers for febrile response, by altering the firing rate of thermoregulation controlling neurons within the hypothalamus.

NSAIDs play a major role in the management of pain in acute and chronic diseases, as well as post surgical pain, and more generally in all those conditions in which pain is associated with inflammation.

Among the NSAIDs, ketoprofen, the salts of ketoprofen, typically the lysin salt, and dexketoprofen (the dextrorotatory stereoisomer of ketoprofen) typically the trometamol salt are among the most active NSAIDs, pertaining to the class of propionic acid derivatives, widely prescribed and available as over-the-counter medication in several countries. It is highly potent and highly effective in relieving pain from traumatic, orthopaedic and rheumatic disorders, in both acute and chronic settings, as well as managing fever, in both children and adults.

In addition to its effects on cyclooxygenase, ketoprofen also reversibly inhibits lipoxygenase, which mediates the conversion of arachidonic acid into leukotrienes, a family of eicosanoid inflammatory mediators. Ketoprofen has also been shown to suppress bradykinin, an inflammation and pain chemical mediator, and to prevent the release of lysosomal enzymes, responsible for the mediation of tissue destruction in inflammatory reactions.

Orally administered ketoprofen is readily absorbed by the gastrointestinal tract, with peak concentration at 0.5-2 hours; it is characterized by a short half-life (1-4 hours), it is rapidly metabolized in the liver and its metabolites excreted in urine, with virtually no bio-accumulation (approximately 80% excretion in 24 h from oral administration).

Interestingly, it has been shown that ketoprofen, as other NSAIDs, has both peripheral and central sites of action, rapidly passing the blood brain barrier, due to its liposolubility.

All these features contribute to a rapid onset of action, flexible dosing, and a reliable tolerance profile.

However, ketoprofen is also characterized by poor solubility and stability in aqueous media, with a water solubility of 0.051 mg/mL at 22° C. and a pKₐ of 4.45. These characteristics, common to most NSAIDs, make it difficult to formulate ketoprofen in pharmaceutical compositions, particularly in liquid dosage forms. In fact, ketoprofen, as most NSAIDs, exerts a chemesthetic (irritant) effect on the oral cavity, throat and pharynx as well as having a bitter taste. Moreover, a bitter taste has also been described as arising from the ingredients used to solubilize NSAIDs and/or reduce their irritant effect.

These problems have been addressed in the art, with several different solutions proposed, for example in U.S. Pat. No. 5,895,789, WO 99/52528, US 2012/0208887, WO 2004/05454, U.S. Pat. No. 5,183,829, and WO 2007/112274.

The applicant has already faced these problems in WO 2005/058276, wherein a pharmaceutical oral dosage form, comprising a NSAID and having good palatability, was disclosed. The composition made use of tromethamine, to solubilize the drug and to eliminate the chemesthetic effect, and glycine, Vitamin B6 or a mixture thereof, to overcome the bitter taste.

EP1974751 discloses a pharmaceutical composition comprising a NSAID, wherein the solubilisation, and the suppression of the chemesthetic effect and of the bitter taste, are achieved using a β-cyclodextrin and tromethamine. A similar composition is disclosed in WO 97/18245, specifically for Naproxen. These applications deal with the problem of masking the NSAIDs bitter taste and chemestetic effect but do not address the technical problem of stability of these solution in the presence of additional ingredients, such as preservatives.

As described, i.e. in the above mentioned patents and patent applications cyclodextrins have been extensively used to enhance the water solubility and stability of hydrophobic drugs, as well as taste masking agents.

Cyclodextrins are cyclic oligosaccharides made of α-D-glucopyranoside units linked via α-(1,4) bonds forming a ring, and the most common are made of 6 (α-cyclodextrin), 7 (β-cyclodextrin), or 8 (γ-cyclodextrin) units. They are characterised by a hydrophobic cavity and a hydrophilic surface, thus are able to entrap a guest molecule by displacing the water molecules present in the cavity forming an inclusion complex.

Without being bound to a specific theory, cyclodextrins have been described as offering a cavity to molecules able to fit within. However, by the term “complex” the Applicant intends to comprise complexes in which one component (the host) forms a cavity containing spaces in the shape of long tunnels or channels in which molecular entities of a second chemical species (the guest) are located (inclusion complexes), or simple combinations of the different components essential for masking the bitter taste and chemestetic effect, which are present in specific molar ratio and which are able to satisfy the technical problem linked to NSAIDs administration and to guarantee a chemico-physical and microbiological stability.

The complexes of the invention are not linked by covalent bonds, the attraction between different molecules being generally due to van der Waals forces, as well as hydrophobic and dipole-dipole interactions. In the case of inclusion complexes, these are a dimensional, geometrically limited fit between the cyclodextrin and the guest molecule, the driving force being the affinity of the hydrophobic guest molecule for the cavity, and the complex stability relying on the number of intermolecular interactions between host and guest.

Despite their wide use as solubility enhancers, cyclodextrins show limited water solubility, β-cyclodextrin being one of the least soluble, with a solubility of 18 mg/mL in water. Such poor solubility is mainly due to the inter- and intra-molecular hydrogen bonds forming between the various hydroxy groups present in the molecule. For this reason, several derivatized cyclodextrins have been synthetized, with various degrees of OH-substitution, in order to tailor their properties both in terms of water solubility and ability to interact with guest molecules.

Among β-cyclodextrin derivatives hydroxypropyl-β-cyclodextrin, an amorphous hydrophilic derivative, shows improved water solubility (600 mg/mL), low toxicity and a satisfactory complexation ability. Sulfobutylether-β-cyclodextrin (SBECD) is also β-cyclodextrin derivative with improved solubility.

As already said, the use of tromethamine, a hydroxyalkylamine, in conjunction with a cyclodextrin to solubilize NSAIDs has been disclosed in EP1974751 and WO 97/18245. Tromethamine is able to stabilize the inclusion complex between the cyclodextrin and the drug, with the formation of a ternary complex wherein tromethamine shows strong intermolecular interactions with both the cyclodextrin and the drug, enhancing not only the drug solubility, but particularly the taste masking action of the complex.

Nevertheless, such ability of the cyclodextrin to interact with tromethamine, while already in a complex with the drug, is also an indication of the cyclodextrin's ability to interact with other suitable substances eventually present in solution.

In fact, it has been shown in the art that cyclodextrins tend to form strong complexes with water soluble polymers, also when already in presence of a drug or other guest molecule (R. S. Hirlekar, et al. Studies on the Effect of Water-Soluble Polymers on Drug-Cyclodextrin Complex Solubility, AAPS PharmSciTech 2009, 10(3), 858-863; T. Loftsson, et al. The effect of water-soluble polymers on the aqueous solubility and complexing abilities of β-cyclodextrin, International Journal of Pharmaceutics 1998, 163(1-2)). The resulting complexes alter the binding constant between the drug and the cyclodextrin, at the same time reducing the concentration of free polymer in solution.

This becomes particularly relevant when attempting to obtain a liquid composition, for instance for oral administration as those of the present invention, or when additional components are added for example when preparing gel composition, with higher viscosity. Liquid pharmaceutical compositions, for oral administration or otherwise, usually contain several pharmaceutically acceptable excipients in order to obtain the desired formulation and to ensure long storage stability as well as micro-biological stability.

Interactions among molecules and macromolecules, such as cyclodextrins is highly unpredictable and difficult to control in terms of nature and amounts of molecules used.

Therefore the obtainment of a composition with good palatability which shows good physico-chemical and microbiological stability during a prolonged storage was not obvious.

### SUMMARY OF THE INVENTION

The Applicant has faced the problem of obtaining liquid compositions for oral administration comprising ketoprofen, having good palatability and improved chemical-physical and microbiological stability.

In particular, the Applicant has faced the problem of obtaining a liquid composition for oral administration comprising a complex of ketoprofen, β-cyclodextrin and an alkylamine having good palatability and improved chemical-physical and micro-biological stability.

In fact the Applicant noted that complexes of ketoprofen, a hydroxyalkylamine and a β-cyclodextrin produced water soluble compositions with good palatability, but unsatisfactory chemical-physical and micro-biological stability. In fact it has been found that the presence of β-cyclodextrins may reduce the activity of some preservatives commonly used in the pharmaceutical field, possibly leading to failure of the challenge test required by the European Pharmacopoeia.

Stability at low temperatures may be also compromised with flocculation and/or precipitation of the complexes.

The Applicant has now surprisingly found that a preservative system consisting of methyl paraben and propyl paraben, in addition to said complexes of ketoprofen was able to ensure the physical, chemical and microbiological stability of the resulting liquid composition after long term storage.

Therefore according to a first aspect the present invention relates to a liquid composition comprising (i) a complex of: a) ketoprofen, dexketoprofen and salts thereof, b) a hydroxylalkylamine and c) a β-cyclodextrin or a derivate, such as hydroxypropyl-β-cyclodextrin or sulfobutylether-β-cyclodextrin (SBECD), wherein said hydroxyalkylamine is selected from the group consisting of: tromethamine, ethanolamine, diethanolamine, triethanolamine, meglumina, 2-amino-2-methyl-1,3-propanediol and 2-amino-1,2,3,-propanetriol, most preferably tromethamine, diethanolamine and triethanolamine, and (ii) a preservative system consisting of methyl paraben and propyl paraben, wherein the amount of ketoprofen or derivative thereof in said solution is equal to or lower than 2% w/V. More preferably, ketoprofen is used at a w/V concentration equal or lower than 1.5%, more preferably at a concentration of from 0.01%-1%, even more preferably at a concentration of from 0.2%-0.8%, comprising the preferred concentration of about 0.5%. Ketoprofen lysin salt and dexketoprofen trometamol concentrations in w/V are adjusted accordingly, on the basis of the Molecular Weight differences. For example, a concentration of 0.5% ketoprofen acid corresponds to about 0.8% w/V ketoprofen lysin salt and to about 0.74% w/V dexketoprofen trometamol.

In molar terms, the complex i) which consists of: a) ketoprofen, dexketoprofen and salts thereof, b) a hydroxyalkylamine and c) a β-cyclodextrin, comprises at least a 3 fold molar ratio of the hydroxyalkylamine and a 0.05-1 molar ratio of the β-cyclodextrin with respect to the active ingredient.

### DETAILED DESCRIPTION OF THE INVENTION

The present invention relates to a liquid composition comprising a complex (i) preferably consisting of: a) ketoprofen, dexketoprofen and salts thereof, b) a hydroxyalkylamine and c) a β-cyclodextrin or a derivative, such as hydroxypropyl-β-cyclodextrin (HP-β-CD) or sulfobutylether-β-cyclodextrin (SBECD), wherein said hydroxyalkylamine is preferably selected from the group consisting of: tromethamine, ethanolamine, diethanolamine, triethanolamine, meglumina, 2-amino-2-methyl-1,3-propanediol and 2-amino-1,2,3,-propanetriol, most preferably tromethamine, diethanolamine and triethanolamine, and (ii) a preservative system consisting of methyl paraben and propyl paraben, wherein the amount of ketoprofen or derivative thereof in said solution is equal to or lower than 2% w/V.

It is intended that, in the following, the term β-cyclodextrin refers to the unmodified as well as to the β-cyclodextrin derivatives mentioned above.

In molar terms, complex i) which preferably consists of: a) ketoprofen, dexketoprofen and salts thereof, b) a hydroxyalkylamine and c) a β-cyclodextrin, comprises at least a 3 molar excess of the hydroxyalkylamine, wherein even more preferably a 3.5 to 6.5 molar ratio is selected and a 0.05-1 molar ratio of the β-cyclodextrin with respect to the active ingredient.

The term ketoprofen in the following, is intended to comprise ketoprofen acids as well as the salts of ketoprofen, preferably the lysin salt, and dexketoprofen (the dextrorotatory stereoisomer of ketoprofen) typically the trometamol salt.

More preferably, ketoprofen is used at a w/V concentration equal or lower than 2%, more preferably at a concentration of from 0.01%-1.5%, even more preferably at a concentration of from 0.2%-1%, comprising the preferred concentration of about 0.5% w/V. Ketoprofen lysin salt and dexketoprofen trometamol concentrations in w/V may be adjusted accordingly, on the basis of the Molecular Weight differences. For example, a concentration of 0.5% ketoprofen acid corresponds to about 0.8% w/V ketoprofen lysin salt and to about 0.74% w/V dexketoprofen trometamol, wherein by the above % w/V is intended the value with respect to the total volume of the composition. Accordingly, a concentration of 2% w/V ketoprofen acid corresponds to 3.6% w/V ketoprofen lysin salt and to about 3% w/V dexketoprofen trometamol.

In the liquid composition according to the present invention the hydroxyalkylamine, preferably selected in the group consisting of: tromethamine, ethanolamine, diethanolamine, triethanolamine, meglumina, 2-amino-2-methyl-1,3-propanediol and 2-amino-1,2,3,-propanetriol, most preferably tromethamine, diethanolamine and triethanolamine, most preferably tromethamine, triethanolamine and diethanolamine, is present at least in a 3-fold molar excess with respect to the active ingredient, or more preferably to at least a 4-fold molar excess, or even more preferably to at least a 5-fold molar excess with respect to the active ingredient.

More preferably, the hydroxyalkylamine is present in a molar excess of at least 6 with respect to the active ingredient, wherein with about 6 we refer to a range comprised from 5.5 to 6.5 molar excess with respect to the active ingredient. Even more preferably, the molar excess of the hydroxyalkylamine versus the active ingredient is comprised from the above mentioned lower values to an upper value of about 10.

Particularly preferred is about a 6-fold molar excess of the hydroxyalkylamine wherein the hydroxyalkylamine is preferably tromethamine.

The term “about” refers to values which comprise the value of interest and +/−a variation of from 1% to 2% of the same mentioned value.

As said above, the liquid composition according to the present invention preferably comprises a β-cyclodextrin or a derivative, in molar ratio of from 0.05 to 1 with respect to the active ingredient. More preferably the molar ratio of β-cyclodextrin or a derivative is comprised of from 0.1 to 0.7 or more preferably of from 0.3 to 0.6. Even more preferably the molar ratio of β-cyclodextrin or a derivative is comprised of from 0.35 to 0.55 with respect of the active ingredient.

Particularly preferred is the composition wherein complex i) comprises about 0.5% w/V ketoprofen the hydroxyalkylamine is tromethamine, the β-cyclodextrin is HP-β-cyclodextrin, present in molar ratios respectively of about: 1/6/0.5.

The above mentioned quantities and molar ratio in the complex allow a good palatability and are stable in solution at 4° C. without precipitation and flocculation after long term storage.

However, only a few preservatives allow a good microbiological stability to the composition of the invention, probably due to interaction with β-cyclodextrin in the complex. As a matter of fact, the Applicant has identified a couple of preservatives that provide long term microbiological stability to the liquid composition without altering the chemico-physical properties.

Therefore, according to these observations the liquid composition of the present invention comprises methyl paraben in an amount of from 0.005 to 1% w/V with respect to the total volume of the composition and preferably comprises also propyl paraben in an amount of from 0.001 to 0.5% w/V with respect to the total volume of the composition.

More preferably, the liquid composition according to the present invention comprises methyl paraben in an amount of from 0.01 to 0.5% w/V with respect to the total volume of the composition.

Even more preferably, the liquid composition according to the present invention comprises methyl paraben in an amount of from 0.1 to 0.3% w/V with respect to the total volume of the composition.

The liquid composition according to the present invention preferably comprises propyl paraben in an amount of from 0.001 to 0.5% w/V with respect to the total volume of the composition.

More preferably, the liquid composition according to the present invention comprises propyl paraben in an amount of from 0.005 to 0.25% w/V with respect to the total volume of the composition.

Even more preferably, the liquid composition according to the present invention comprises propyl paraben in an amount of from 0.01 to 0.1% w/V with respect to the total volume of the composition.

Furthermore, according to a preferred embodiment of the present invention, the liquid composition comprises a methyl paraben: propyl paraben weight ratio of from about 10:1 to about 1:1, more preferably from about 8:1 to about 2:1.

Most preferably, the liquid composition comprises a methyl paraben: propyl paraben weight ratio of from about 6:1 to about 4:1.

Preferably, the liquid pharmaceutical composition according to the present invention is an aqueous formulation for oral administration.

Preferably, the liquid pharmaceutical composition according to the present invention is prepared in suitable dosage forms, such as for example solutions, suspensions, syrups, gels, and spray. More preferably, said dosage form is a solution or a gel. Even more preferably, said dosage form is a viscous solution or gel.

Water is preferably used as the main solvent for the liquid pharmaceutical composition of the present invention, in particular demineralized water, purified water, distilled water, and the like.

The pharmaceutical composition according to the present invention may comprise other pharmaceutically acceptable ingredients and/or excipients.

The term pharmaceutically acceptable excipient is understood to comprise without any particular limitations any material which is suitable for the preparation of a liquid pharmaceutical composition which is to be administered to a living being, such as, for example co-solvents, stabilizers, antioxidants, pH correctors, buffers, surfactants, chelating agent, colorants, flavouring agents, sugars, sweeteners, and/or perfumes.

Advantageously, the liquid pharmaceutical composition of the present invention comprises one or more flavouring agent, such as, for example, grapefruit flavour, raspberry flavour, lemon flavour, orange flavour, caramel flavour, vanilla flavour, cream flavour, and the like.

Advantageously, the liquid pharmaceutical composition of the present invention comprises one or more sweetener, such as, for example, aspartame, saccharin, acesulfame, sucralose, and the like.

Advantageously, the liquid pharmaceutical composition of the present invention comprises one or more sugar, such as, for example, lactose, glucose, sucrose, and the like.

Advantageously, the liquid pharmaceutical composition of the present invention comprises one or more chelating agent, such as, for example, diethylenetriaminepentaacetic acid (DTPA), ethylenedinitrilotetraacetic acid, (EDTA), nitrilotriacetic acid (NTA), and the like.

Preferably, the liquid pharmaceutical composition of the present invention comprises one or more co-solvent selected from the group of glycols and polyols, such as, for example, glycerol, propylene glycol, 1,3-butylene glycol, and the like.

The pH of the aqueous composition, which has to be orally administered, is preferably close to neutrality, i.e. comprised of from 5 to 8, preferably 5.2-7.5 more preferably 5.5-6.5.

In a preferred embodiment, the liquid pharmaceutical composition of the present invention is an aqueous gel comprising a viscosity modifier.

Preferably, the viscosity modifier is a hydrophilic polymer selected from the group consisting of alginates, carbomers, polyacrylates, cellulose derivatives, such as hydroxyethyl, hydroxypropyl and carboxymethylcellulose, gums, such as xanthan gum, guar gum, proteins, such as gelatine and pectin, and high molecular weight polysaccharides such as carrageenan.

In the so obtained aqueous gel composition, the viscosity modifier is present in an amount of from 0.01 to 1.0% w/V with respect to the total volume of the pharmaceutical composition. Even more preferably the viscosity modifier is in an amount of from 0.20 to 0.80% w/V with respect to the total volume of the composition and even more preferably it is comprised of from 0.30 to 0.50% w/V.

In the gel compositions the additional presence of a viscosity modifier, typically a polymer, requires further evaluation of stability as better detailed in the experimental part. Therefore, according to this preferred embodiment, complex i) consists of: a) ketoprofen, dexketoprofen or salts thereof, b) a hydroxyalkylamine and c) a β-cyclodextrin, in at least a 3-fold molar ratio, preferably a 3-7 molar ratio, even more preferably a 3.5 to 6.5 molar ratio of the hydroxyalkylamine, together with a β-cyclodextrin molar ratio generally lower than in the liquid compositions, i.e. of from 0.08-0.4, more preferably 0.1-0.4, even more preferably of about a 0.35 molar ratio, with respect to the active ingredient ketoprofen or a derivative thereof.

The active ingredient is equal to or lower than 2% w/V. More preferably, ketoprofen is ketoprofen acid and is used at a w/V concentration equal or lower than 1.5%, more preferably at a concentration of from 0.01%-1%, even more preferably at a concentration of from 0.2%-0.8% w/V, comprising the preferred concentration of about 0.5% wherein the above % w/V concentrations are intended to comprise the upper and lower limit of the range and are referred to the total volume of the final composition. Ketoprofen lysin salt and dexketoprofen trometamol concentrations in w/V will be adjusted accordingly, on the basis of the Molecular Weight differences. For example, a concentration of 0.5% w/V ketoprofen acid corresponds to about 0.8% w/V ketoprofen lysin salt and to a 0.74% w/V dexketoprofen trometamol.

The gel compositions further comprise the preservative system defined above with the same qualities and preferred quantities. According to a preferred embodiment, the composition comprises a viscosity modifier and complex i) comprises or preferably consists of, about 0.5% w/V of ketoprofen, the hydroxyalkylamine is tromethamine, the β-cyclodextrin is 2-HP-β-cyclodextrin, wherein the active principle/hydroxyalkylamine and β-cyclodextrin are in molar ratios respectively of about: 1/6/0.35.

Preferably, the liquid pharmaceutical composition of the present invention is characterized by a viscosity equal to or higher than 1 mPa*s and preferably equal to or lower than 2000 mPa*s. Even more preferably, the liquid pharmaceutical composition of the present invention is characterized by a viscosity of from 500 mPa*s to 1500 mPa*s. Most preferably, the liquid pharmaceutical composition of the present invention is characterized by a viscosity of about 1000 mPa*s.

### EXPERIMENTAL EXAMPLES

### Materials

| Substance | PM | Fornitore | Product code |
|---|---|---|---|
| Ketoprofen (acid) | 254.281 | Jiuzhou | 2014-0009 |
| | | Cosma S.p.A. | 1081 |
| Trometamol | 121.14 | Merck | 1.08386.1000 |
| (EMKPROVE) | | | |
| HP-beta-CD | 1400 | Roquette | 346112100 |
| SBECD (sodium salt) | 2163 | Captisol | RC-BSF-005 |
| Beta-CD | 1135 | Roquette | 341001114 |

### Example 1—Palatability Test

Five aqueous solutions of ketoprofen (0.5 w/V %) and increasing amounts of tromethamine were prepared and subjected to a palatability test to assess the tromethamine ability of masking both the chemesthetic effect and the bitter taste of the ketoprofen.

The amounts of tromethamine contained in aqueous solutions 1 to 5 are described in the Table 1 below.

TABLE 1

| Sample | Tromethamine (w/V %) |
|---|---|
| 1 | 0.5 |
| 2 | 1 |
| 3 | 1.5 |
| 4 | 2 |
| 5 | 3 |

The irritation of the oral mucosae by the NSAIDs shows great individual variability, therefore the panel of individuals for the palatability test had to be properly selected. Indeed, whereas for some individuals the irritation may be “slightly noticeable”, others define it as “strong” or “very strong” (Breslin et al. “Ibuprofen as a chemesthetic stimulus: evidence of a novel mechanism of throat irritation”, Chem. Sens. 26: 55-65, 2001). In order to select only those individuals clearly sensitive to the irritant action of the NSAIDs, a preliminary test was performed administering an aqueous solution containing 0.5% w/V of ketoprofen acid.

40 individuals between 20 and 40 years old were requested to follow the standard procedure described hereinbelow when taking the solution: —sip 10 ml of demineralized water, hold it in the mouth for 10 seconds and then swallow it, —sip 10 ml of solution, hold it in the mouth for 10 seconds and then swallow it.

Indications were given for correctly defining the perceived irritant stimuli, as follows:

| Stimulus | Description |
|---|---|
| Burning | Sensation generated by abrasion of the skin or by |
| | exposure to high temperature, or to the irritant action of |
| | alcohol |
| Stinging | Brief sensation produced as from an insect bite or from |
| | thorns |
| Prickling | Sensation similar to that caused by the action of small |
| | penetrating needles |
| Numbness | Diffuse sensation similar to the start of action of an |
| | anaesthetic (not an absence of sensation) |

These 40 individuals were then asked to evaluate the intensity of the irritation in the oral cavity, taking into consideration each stimulus described above, at time 0, at 30 seconds, 1 minute and 5 minutes after the administration, and 3 points were assigned to those who defined the sensation as “strong”, 2 points to those who defined the sensation as “moderate”, 1 point to those who defined the sensation as “mild” and 0 points to those who defined the solution as provoking no irritant sensation.

Only those individuals who showed greater sensitivity (more than 40 points in total) towards the unpleasant sensations generated by ketoprofen were thus selected.

Solutions 1 to 5 were then administered to the 20 selected individuals, following the same procedure and assigning the points as described above.

In this case more evaluation time points were used, as the 20 individuals were requested to evaluate the intensity of the irritation in the mouth and the perceived taste at time 0, 30 seconds, 1 minute, 2 minutes, 3 minutes, 5 minutes, 10 minutes and 15 minutes after the administration.

The sum of the evaluations (0-15 minutes) for, respectively, the burning, the stinging, the prickling and the numbness was calculated for each individual, along with the sum of the evaluations (0-15 minutes) for all the sensations.

The individuals were also asked to describe the bitter taste perceived, with 3 points assigned to those who described the bitter taste as “strong”, 2 points to those who described the bitter taste as “moderate”, and 1 point to those who described the bitter taste as “mild”.

These parameters were analysed by the Wilcoxon “signed rank” method to compare the solutions. The final scores are shown in the following Table 2.

TABLE 2

| Sample | Tromethamine (w/V %) | Chemesthetic effect | Bitter taste |
|---|---|---|---|
| 1 | 0.5 | Yes 3 | Yes 2 |
| 2 | 1 | No 1 | Yes 2 |
| 3 | 1.5 | No 0 | Yes 2 |
| 4 | 2 | No 0 | Yes 2 |
| 5 | 3 | No 0 | Yes 2 |

As evident from the results summarised in Table 2 the solution containing 1% of tromethamine was already completely void of chemesthetic effect. However, all the samples were described as having bitter taste, even at 3% tromethamine.

### Example 2—Stability Test

Solutions 2 to 5, which proved void of chemesthetic effect in the preceding example 1, were subjected to a chemical-physical stability test to verify the absence of precipitation and/or flocculation by maintaining a sample at low temperature (4° C.) for 3 months.

TABLE 3

| Sample | Stability |
|---|---|
| 2 | No |
| 3 | Yes |
| 4 | Yes |
| 5 | Yes |

Table 3 shows that tromethamine should be present in an amount >1% to maintain the active ingredient in solution in the long term, at 4° C., as demonstrated by the results of the above Table 3.

Example 3—Palatability Test Four aqueous solutions (solutions 6-8) containing 0.5% of ketoprofen, 1.5% of tromethamine and increasing amounts of 2-hydroxypropyl-β-cyclodextrin (2HP-β-CD) were prepared and subjected to a palatability test to assess the 2HP-β-CD ability of eliminating the bitter taste from the solution.

The test was performed as already described in the example 1 above, and the results, together with the amounts of 2HP-β-CD contained in aqueous solutions 6 to 9, are summarized in the Table 4 below.

TABLE 4

| Sample | 2HP-β-CD (w/V %) | Bitter taste |
|---|---|---|
| 6 | 0.5 | Yes |
| 7 | 1 | No |
| 8 | 2.5 | No |
| 9 | 5 | No |

The results summarised in Table 4 clearly show that the minimum amount of 2HP-β-CD required to completely eliminate the bitter taste should be higher than 0.5%.

### Example 4—Microbiological Stability Tests

To select the proper preservative system, able to ensure microbiological stability to the composition, six different preservative systems, consisting of six different couples of preservatives, were tested in the composition described in the following Table 5.

TABLE 5
Liquid composition

| Ingredient | Amount (% w/V) |
|---|---|
| Ketoprofen | 0.5 |
| Tromethamine | 1.5 |
| 2HP-β-CD | 1 |
| Preservative system | As for Table 6 |
| Propylene glycol | 2.5 |
| Sugar | 60 |
| Citric acid monohydrate | 0.4 |
| Titriplex | 0.1 |
| flavour | 0.2 |
| Demineralized water | To reach 100 mL |

The different couples of preservatives are described in the Table 6 below.

TABLE 6

| Liquid composition | Preservative | Amount (% w/V) |
|---|---|---|
| 10 | Sodium benzoate | 0.5 |
| | Potassium sorbate | 0.18 |
| 11 | Methyl paraben | 0.2 |
| | Potassium sorbate | 0.18 |
| 12 | Sodium benzoate | 0.5 |
| | Propyl paraben | 0.04 |
| 13 | Methyl paraben | 0.2 |
| | Propyl paraben | 0.051 |
| 14 | Ethyl paraben | 0.15 |
| | Propyl paraben | 0.05 |
| 15 | Methyl paraben | 0.25 |
| | Ethyl paraben | 0.1 |

Liquid compositions 10 to 15 were thus subjected to a preservatives efficacy test (challenge test) according to the European Pharmacopoeia (VIII^{th} edition).

The liquid compositions were tested against four bacteria: Escherichia coli, Pseudomonas aeruginosa, Staphylococcus aureus, and Burkholderia cepacia, and three fungi: Candida albicans, Aspergillus brasiliensis, and Zygosaccharomyces rouxii.

20 g aliquots of each composition 10 to 15 were put in TSA culture media, for samples to be inoculated with bacteria, or in SDA culture media, for samples to be inoculated with fungi, and kept at 20-25° C.

Samples were first evaluated for the presence of any microorganisms or pathogens that may have been introduced during the manufacturing process. Then, each sample was inoculated with 200 μL of a different microorganism and incubated for up to 28 days at 30-35° C. for samples inoculated with bacteria and at 20-25° C. for samples inoculated with fungi.

All samples were analysed immediately after inoculation (time 0), and at 14 and 28 days of incubation, to assess the number of viable bacteria or fungal cells per mL of sample (CFU/mL).

For any of the four bacteria tested, preservative challenge testing requires not less than a 3.0 log reduction in microbial concentration from the initial count by day 14, and no increase in microbial concentration levels at day 28 over those measured at day 14. For any of the three fungi, it is required not less than a 1.0 log reduction in microbial concentration from the initial count by day 14, and no increase in microbial concentration levels at day 28. A composition is considered compliant only when it shows positive results for all the microorganisms tested.

Table 7 below shows the results obtained for each liquid composition 10 to 15.

TABLE 7

| Liquid composition | Challenge test |
|---|---|
| 10 | Not compliant |
| 11 | Not compliant |
| 12 | Not compliant |
| 13 | Compliant |
| 14 | Compliant |
| 15 | Compliant |

Only compositions 13 to 15, containing respectively methyl paraben/propyl paraben, ethyl paraben/propyl paraben, and methyl paraben/ethyl paraben, where able to pass the challenge test. Subsequently, the three selected couple of preservatives were tested in compositions with the same ingredients as those described in the above Table 5, but containing increasing amounts of 2HP-β-CD, as described in the Table 8 below.

TABLE 8

| | Methyl | Ethyl | Propyl | |
|---|---|---|---|---|
| | paraben | paraben | paraben | 2HP-β-CD |
| Composition | (% w/V*) | (% w/V*) | (% w/V*) | (% w/V) |
| 16 | 0.2 | — | 0.051 | 1.5 |
| 17 | — | 0.15 | 0.05 | 1.5 |
| 18 | 0.25 | 0.1 | — | 1.5 |
| 19 | 0.2 | — | 0.051 | 2.5 |
| 20 | — | 0.15 | 0.05 | 2.5 |
| 21 | 0.25 | 0.1 | — | 2.5 |

*concentration in accordance with the EMA indications

A challenge test was performed following the same procedure described above, and the results are summarized in Table 9 below.

TABLE 9

| Liquid composition | 2HP-β-CD (% w/V) | Challenge test |
|---|---|---|
| 16 | 1.5 | Compliant |
| 17 | ″ | Compliant |
| 18 | ″ | Compliant |
| 19 | 2.5 | Not compliant |
| 20 | ″ | Not compliant |
| 21 | ″ | Not compliant |

As evident from Table 9, 2HP-β-CD in concentrations equal or higher than 2.5% w/V negatively interacts with the preservative system, rendering the composition not compliant.

### Example 5—Physical Stability Test at 4° C.

Liquid compositions 13 to 18 described in the preceding example 4 were then subjected to a chemical-physical stability test to verify the absence of precipitation and/or flocculation by maintaining a sample at low temperature (4° C.) for 3 months.

TABLE 10

| Liquid composition | Stability test |
|---|---|
| 13 | Yes |
| 14 | No |
| 15 | No |
| 16 | Yes |
| 17 | No |
| 18 | No |

Only compositions 13 and 16, containing methyl paraben/propyl paraben as preservative system, showed sufficient stability.

The preservative system consisting of methyl paraben/propyl paraben was therefore the only one that proved able to ensure at the same time microbiological and physical-chemical stability for a liquid composition containing 0.5% of ketoprofen, 1.5% of tromethamine and from 1 to 1.5% of 2HP-β-CD.

### Example 6—Microbiological Stability Tests

Aqueous gel compositions 22 and 23 containing respectively 1 and 1.5% of 2HP-β-CD, were prepared and their composition is described in the following Table 11.

TABLE 11

| | Aqueous gel 22 | Aqueous gel 23 |
|---|---|---|
| Ingredient | Amount (% w/V) | Amount (% w/V) |
| Ketoprofen | 0.5 | 0.5 |
| Tromethamine | 1.5 | 1.5 |
| 2HP-β-CD | 1 | 1.5 |
| Methyl paraben | 0.2 | 0.2 |
| Propyl paraben | 0.05 | 0.05 |
| Xanthan gum | 0.4 | 0.4 |
| Propylene glycol | 2.5 | 2.5 |
| Sugar | 60 | 60 |
| Citric acid monohydrate | 0.31 | 0.31 |
| Titriplex | 0.1 | 0.1 |
| flavour | 0.17 | 0.17 |
| Demineralized water | To 100 mL | To 100 mL |

A challenge test was performed following the same procedure described in example 4 above, and the results are summarized in Table 12 below.

TABLE 12

| Aqueous gel | Challenge test |
|---|---|
| 22 | Compliant |
| 23 | Not compliant |

As evident from Table 12, the addition of an hydrophilic polymer such as xanthan gum, required to obtain a viscous composition, affects the microbiological stability of the resulting composition. In the above example 2HP-β-CD at 1.5% w/V concentration, seems to interact with the polymer, possibly also with the preservative system, rendering the composition not compliant.

The maximum amount of 2HP-β-CD to be used in conjunction with the preservative systems selected in an aqueous gel, in order to ensure microbiological stability, should be lower than 1.5%. % w/V adjustment with respect to the different molecular weight of cyclodextrins are carried out as known in the art.

### Example 7—Preparative Example—Liquid Compositions

Liquid composition according to the present invention can be represented by compositions described in Table 13 and below, where different 1-CDs, such as sulphobutylether-β-cyclodextrin (SBECD), 2 Hydroxypropyl-β-cyclodextrin (2 HP-β-CD) and β-cyclodextrin (β-CD) and different salts or enantiomeric form of ketoprofen have been used.

TABLE 13
Liquid composition 24

| Ingredient | Amount (% w/V) |
|---|---|
| Ketoprofen | 0.5 |
| Tromethamine | 1.5 |
| 2HP-β-CD | 1.5 |
| Methyl paraben | 0.24 |
| Propyl paraben | 0.04 |
| Propylene glycol | 2.5 |
| Flavour | 0.75-1 |
| Sugar | 60 |
| Citric acid monohydrate | 0.75 |
| EDTA | 0.1 |
| Demineralized water | qs 100 mL |

TABLE 13.1
Liquid composition 25

| Ingredient | Amount (% w/V) |
|---|---|
| Ketoprofen | 0.5 |
| Tromethamine | 1.5 |
| SBECD | 2.3 |
| Methyl paraben | 0.24 |
| Propyl paraben | 0.04 |
| Propylene glycol | 2.5 |
| Flavour | 0.75-1 |
| Sugar | 60 |
| Citric acid monohydrate | 0.75 |
| EDTA | 0.1 |
| Demineralized water | qs 100 mL |

TABLE 13.2
Liquid composition 26

| Ingredient | Amount (% w/V) |
|---|---|
| Ketoprofen | 0.5 |
| Tromethamine | 1.5 |
| β-CD | 1.17 |
| Methyl paraben | 0.24 |
| Propyl paraben | 0.04 |
| Propylene glycol | 2.5 |
| Flavour | 0.75-1 |
| Sugar | 60 |
| Citric acid monohydrate | 0.75 |
| EDTA | 0.1 |
| Demineralized water | qs 100 mL |

TABLE 13.3
Liquid composition 27

| Ingredient | Amount (% w/V) |
|---|---|
| Ketoprofen lysin salt | 0.8 |
| Tromethamine | 1.5 |
| 2HP-β-CD | 1.5 |
| Methyl paraben | 0.24 |
| Propyl paraben | 0.04 |
| Propylene glycol | 2.5 |
| Flavour | 0.75-1 |
| Sugar | 60 |
| Citric acid monohydrate | 0.75 |
| EDTA | 0.1 |
| Demineralized water | qs 100 mL |

TABLE 13.4
Liquid composition 28

| Ingredient | Amount (% w/V) |
|---|---|
| Ketoprofen lysin salt | 0.8 |
| Tromethamine | 1.5 |
| SBECD | 2.3 |
| Methyl paraben | 0.24 |
| Propyl paraben | 0.04 |
| Propylene glycol | 2.5 |
| Flavour | 0.75-1 |
| Sugar | 60 |
| Citric acid monohydrate | 0.75 |
| EDTA | 0.1 |
| Demineralized water | qs 100 mL |

TABLE 13.5
Liquid composition 29

| Ingredient | Amount (% w/V) |
|---|---|
| Ketoprofen lysin salt | 0.8 |
| Tromethamine | 1.5 |
| β-CD | 1.17 |
| Methyl paraben | 0.24 |
| Propyl paraben | 0.04 |
| Propylene glycol | 2.5 |
| Flavour | 0.75-1 |
| Sugar | 60 |
| Citric acid monohydrate | 0.75 |
| EDTA | 0.1 |
| Demineralized water | qs 100 mL |

TABLE 13.6
Liquid composition 30

| Ingredient | Amount (% w/V) |
|---|---|
| Dexketoprofen trometamol | 0.738 |
| Tromethamine | 1.5 |
| 2HP-β-CD | 1.5 |
| Methyl paraben | 0.24 |
| Propyl paraben | 0.04 |
| Propylene glycol | 2.5 |
| Flavour | 0.75-1 |
| Sugar | 60 |
| Citric acid monohydrate | 0.75 |
| EDTA | 0.1 |
| Demineralized water | qs 100 mL |

TABLE 13.7
Liquid composition 31

| Ingredient | Amount (% w/V) |
|---|---|
| Dexketoprofen trometamol | 0.738 |
| Tromethamine | 1.5 |
| SBECD | 2.3 |
| Methyl paraben | 0.24 |
| Propyl paraben | 0.04 |
| Propylene glycol | 2.5 |
| Flavour | 0.75-1 |
| Sugar | 60 |
| Citric acid monohydrate | 0.75 |
| EDTA | 0.1 |
| Demineralized water | qs 100 mL |

TABLE 13.8
Liquid composition 32

| Ingredient | Amount (% w/V) |
|---|---|
| Dexketoprofen trometamol | 0.738 |
| Tromethamine | 1.5 |
| β-CD | 1.17 |
| Methyl paraben | 0.24 |
| Propyl paraben | 0.04 |
| Propylene glycol | 2.5 |
| Flavour | 0.75-1 |
| Sugar | 60 |
| Citric acid monohydrate | 0.75 |
| EDTA | 0.1 |
| Demineralized water | qs 100 mL |

### Example 8—Preparative Example—Aqueous Gel

Gel compositions according to the present invention were prepared as described in Table 14 and below.

TABLE 14
Gel composition 33

| Ingredient | Amount (% w/V) |
|---|---|
| Ketoprofen | 0.5 |
| Tromethamine | 1.5 |
| 2HP-β-CD | 1 |
| Methyl paraben | 0.24 |
| Propyl paraben | 0.04 |
| Propylene glycol | 2.5 |
| Xanthan Gum | 0.4 |
| Flavour | 0.75-1 |
| Sugar | 60 |
| Citric acid monohydrate | 0.75 |
| EDTA | 0.1 |
| Demineralized water | qs 100 mL |

TABLE 14.1
Gel composition 34

| Ingredient | Amount (% w/V) |
|---|---|
| Ketoprofen | 0.5 |
| Tromethamine | 1.5 |
| SBECD | 1.54 |
| Methyl paraben | 0.24 |
| Propyl paraben | 0.04 |
| Propylene glycol | 2.5 |
| Xanthan Gum | 0.4 |
| Flavour | 0.75-1 |
| Sugar | 60 |
| Citric acid monohydrate | 0.75 |
| EDTA | 0.1 |
| Demineralized water | qs 100 mL |

TABLE 14.2
Gel composition 35

| Ingredient | Amount (% w/V) |
|---|---|
| Ketoprofen | 0.5 |
| Tromethamine | 1.5 |
| β-CD | 0.78 |
| Methyl paraben | 0.24 |
| Propyl paraben | 0.04 |
| Propylene glycol | 2.5 |
| Xanthan Gum | 0.4 |
| Flavour | 0.75-1 |
| Sugar | 60 |
| Citric acid monohydrate | 0.75 |
| EDTA | 0.1 |
| Demineralized water | qs 100 mL |

TABLE 14.3
Gel composition 36

| Ingredient | Amount (% w/V) |
|---|---|
| Ketoprofen lysin salt | 0.8 |
| Tromethamine | 1.5 |
| 2HP-β-CD | 1 |
| Methyl paraben | 0.24 |
| Propyl paraben | 0.04 |
| Propylene glycol | 2.5 |
| Xanthan Gum | 0.4 |
| Flavour | 0.75-1 |
| Sugar | 60 |
| Citric acid monohydrate | 0.75 |
| EDTA | 0.1 |
| Demineralized water | qs 100 mL |

TABLE 14.4
Gel composition 37

| Ingredient | Amount (% w/V) |
|---|---|
| Ketoprofen lysin salt | 0.8 |
| Tromethamine | 1.5 |
| SBECD | 1.54 |
| Methyl paraben | 0.24 |
| Propyl paraben | 0.04 |
| Propylene glycol | 2.5 |
| Xanthan Gum | 0.4 |
| Flavour | 0.75-1 |
| Sugar | 60 |
| Citric acid monohydrate | 0.75 |
| EDTA | 0.1 |
| Demineralized water | qs 100 mL |

TABLE 14.5
Gel composition 38

| Ingredient | Amount (% w/V) |
|---|---|
| Ketoprofen lysin salt | 0.8 |
| Tromethamine | 1.5 |
| β-CD | 0.78 |
| Methyl paraben | 0.24 |
| Propyl paraben | 0.04 |
| Propylene glycol | 2.5 |
| Xanthan Gum | 0.4 |
| Flavour | 0.75-1 |
| Sugar | 60 |
| Citric acid monohydrate | 0.75 |
| EDTA | 0.1 |
| Demineralized water | qs 100 mL |

TABLE 14.6
Gel composition 39

| Ingredient | Amount (% w/V) |
|---|---|
| Dexketoprofen trometamol | 0.738 |
| Tromethamine | 1.5 |
| 2HP-β-CD | 1 |
| Methyl paraben | 0.24 |
| Propyl paraben | 0.04 |
| Propylene glycol | 2.5 |
| Xanthan Gum | 0.4 |
| Flavour | 0.75-1 |
| Sugar | 60 |
| Citric acid monohydrate | 0.75 |
| EDTA | 0.1 |
| Demineralized water | qs 100 mL |

TABLE 14.7
Gel composition 40

| Ingredient | Amount (% w/V) |
|---|---|
| Dexketoprofen trometamol | 0.738 |
| Tromethamine | 1.5 |
| SBECD | 1.54 |
| Methyl paraben | 0.24 |
| Propyl paraben | 0.04 |
| Propylene glycol | 2.5 |
| Xanthan Gum | 0.4 |
| Flavour | 0.75-1 |
| Sugar | 60 |
| Citric acid monohydrate | 0.75 |
| EDTA | 0.1 |
| Demineralized water | qs 100 mL |

TABLE 14.8
Gel composition 41

| Ingredient | Amount (% w/V) |
|---|---|
| Dexketoprofen trometamol | 0.738 |
| Tromethamine | 1.5 |
| β-CD | 0.78 |
| Methyl paraben | 0.24 |
| Propyl paraben | 0.04 |
| Propylene glycol | 2.5 |
| Xanthan Gum | 0.4 |
| Flavour | 0.75-1 |
| Sugar | 60 |
| Citric acid monohydrate | 0.75 |
| EDTA | 0.1 |
| Demineralized water | qs 100 mL |

TABLE 14.9
Gel composition 42

| Ingredient | Amount (% w/V) |
|---|---|
| Ketoprofen | 1 |
| Tromethamine | 1.5 |
| 2HP-β-CD | 0.5 |
| Methyl paraben | 0.24 |
| Propyl paraben | 0.04 |
| Propylene glycol | 2.5 |
| Xanthan Gum | 0.4 |
| Flavour | 0.75-1 |
| Sugar | 60 |
| Citric acid monohydrate | 0.6 |
| EDTA | 0.1 |
| Demineralized water | qs 100 mL |

TABLE 14.10
Gel composition 43

| Ingredient | Amount (% w/V) |
|---|---|
| Ketoprofen | 1 |
| Tromethamine | 1.5 |
| SBECD | 0.77 |
| Methyl paraben | 0.24 |
| Propyl paraben | 0.04 |
| Propylene glycol | 2.5 |
| Xanthan Gum | 0.4 |
| Flavour | 0.75-1 |
| Sugar | 60 |
| Citric acid monohydrate | 0.6 |
| EDTA | 0.1 |
| Demineralized water | qs 100 mL |

### Example 9—Long Term Physical, Chemical and Microbiological Stability

Stable compositions resulting from the preliminary assays were tested according to the ICH Q1A “Stability testing of new drug substances and products” Guidelines, in the following ICH conditions:
25° C./60% RH
30° C./65% RH
40° C./75% RH

Results at 6 months are shown in Table 15:

TABLE 15

| | | | 6 months | | |
|---|---|---|---|---|---|
| | | | 25° C./ | 30° C./ | 40° C./ |
| Test | Specifications | Initial | 60% RH | 65% RH | 75% RH |
| Appearance | Clear to slightly | Complies | Complies | Complies | Complies |
| | opalescent | | | | |
| Ketoprofen | 95%-105% | Complies | Complies | Complies | Complies |
| Assay | | | | | |
| Preservatives | 80-110% | Complies | Complies | Complies | Complies |
| Assay | | | | | |
| Microbiological | TAMC ≤ 10²/ml | Complies | Complies | Complies | Complies |
| Quality (Eur. Ph.) | TYMC ≤ 10/ml | | | | |
| | E. Coli/ml = absent | | | | |
| | Preservative | Complies | Complies | Complies | Complies |
| | efficacy test | | | | |

The assay was positive for all the conditions tested and for all the parameters, indicating that the active ingredient in solution is stable at 6 months, also in the gel composition to which Table 15 refers, the preservative was maintained and the solution was free from any contamination, as reported above.

The compositions were also evaluated at 12 and 18 months under ICH conditions. At 12 months they were stable in the conditions of 30° C. and 65% humidity (30° C./65% RH) and at 18 months in the conditions of 25° C. and 60% humidity (25° C./60% RH).

In summary, stability of the physico-chemical and microbiological properties of the composition according to the invention was assessed up to 18 months.

Stability was preliminarly assessed also for gel formulations with API concentration of 1% and evaluated by:
a predictive stress of physical stability (4° C. for 1 month): the result was compliant with the product specifications,
a predictive stress of chemical stability (50° C. for 1 month): the result was compliant with the product specification, also if compared with the formulation under ICH stability, stressed at the same conditions (50° C. for 1 month) and
a microbiologial tests (Ph. Eur) to ensure the microbiological quality and the preservation of the formulations.

Gel formulations were demonstrated to be compliant and stable in the above assay conditions.

1: An aqueous composition comprising (i) a complex consisting of a) ketoprofen or derivatives selected from the group consisting of salts and enantiomers thereof, b) a hydroxyalkylamine and c) a β-cyclodextrin and (ii) a preservative system consisting of methyl paraben and propyl paraben, where in the complex i), the hydroxyalkylamine is present in a molar ratio of at least 3 and the β-cyclodextrin in a molar ratio of from 0.05-1 with respect to the active ingredient and the active ingredient is in amount equal to or lower than 2% w/V.

2: The aqueous composition according to claim 1 wherein said ketoprofen derivatives are selected in the group consisting of: dexketoprofen, dexketoprofen tromethamol and ketoprofen lysin salt.

3: The aqueous composition according to claim 1, wherein the hydroxyalkylamine is selected in the group consisting of: tromethamine, ethanolamine, diethanolamine, triethanolamine, meglumina, 2-amino-2-methyl-1,3-propanediol and 2-amino-1,2,3,-propanetriol.

4: The aqueous composition according to claim 3 wherein the hydroxylakylamine is selected from the group consisting of: tromethamine, diethanolamine and triethanolamine.

5: The aqueous composition according to claim 3, wherein the hydroxyalkylamine is present in complex i) in a molar ratio of at least 4 with respect to the active ingredient.

6: The aqueous composition according to claim 3, wherein the hydroxyalkylamine is present in complex i) in a molar ratio of from 3 to 7 with respect to the active ingredient.

7: The aqueous composition according to claim 1, wherein the β-cyclodextrin is selected from: 2-HP-β-cyclodextrin and sulfobutyl ether-β-cyclodextrin.

8: The aqueous composition according to claim 1, wherein the β-cyclodextrin is 2-HP-β-cyclodextrin.

9: The aqueous composition according to claim 1, wherein the β-cyclodextrin is in a molar ratio of from 0.1 to 0.7 with respect to the active ingredient.

10: The aqueous composition according to claim 1, wherein the molar ratio of β-cyclodextrin in complex i) is of from 0.08 to 0.4 with respect to the active ingredient.

11: The aqueous composition according to claim 10, further comprising a viscosity modifier.

12: The aqueous composition according to claim 11 wherein the viscosity modifier is a hydrophilic polymer selected from the group consisting of: alginates, carbomers, polyacrylates, cellulose derivatives, such as hydroxyethyl, hydroxypropyl- and carboxymethyl-cellulose, gums, such as xanthan gum, guar gum, proteins and high molecular weight polysaccharides.

13: The aqueous composition according to claim 12 wherein said proteins are selected from gelatine and pectin.

14: The aqueous composition according to claim 12, wherein said high molecular weight polysaccharide is carrageenan.

15: The aqueous composition according to claim 11, wherein said viscosity modifier is present in an amount of from 0.01 to 1.0% w/V with respect to the total volume of the pharmaceutical composition.

16: The aqueous composition according to claim 1, wherein methyl paraben in present in an amount of from 0.005 to 1% w/V with respect to the total volume of the composition.

17: The aqueous composition according to claim 1, wherein methyl paraben in present in an amount of from 0.01 to 0.5% w/V with respect to the total volume of the composition.

18: The aqueous composition according to claim 16, wherein propyl paraben is in amount of from 0.001 to 0.5% w/V with respect to the total volume of the composition.

19: The aqueous composition according to claim 18 wherein propyl paraben is in amount of from 0.005 to 0.25% w/V with respect to the total volume of the composition.

20: The aqueous composition according to claim 1, wherein the pH is comprised of from 5 to 8.
